(19) United States
(12) Patent Application Publication
(10) Pub. No.: US 2024/0374154 A1
(43) Pub. Date: Nov. 14, 2024

(54) ARTERIAL PULSE WAVE MEASUREMENT SYSTEM AND PULSE WAVE MEASUREMENT METHOD

(71) Applicant: AUO Corporation, Hsinchu (TW)

(72) Inventors: Jung-Teng Pan, Hsinchu (TW); Chin-Tang Chuang, Hsinchu (TW); Wen Ching Kao, Hsinchu (TW)

(73) Assignee: AUO Corporation, Hsinchu (TW)

(21) Appl. No.: 18/453,278

(22) Filed: Aug. 21, 2023

(30) Foreign Application Priority Data
May 12, 2023 (TW) 112117709

(51) Int. Cl. A61B 5/022; A61B 5/0225

(52) U.S. Cl. CPC A61B 5/02233; A61B 5/0225; A61B 2562/04

(57) ABSTRACT

An arterial pulse wave measurement system and a pulse wave measurement method are provided. The system includes a first sensor, a pressurizing element, and a processing device. The first sensor is arranged on the radial artery to detect a pulse wave amplitude of a radial artery. The pressurizing element contacts the first sensor to pressurize the first sensor. The processing device is coupled to the first sensor and the pressurizing element so as to control the first sensor to continuously detect the pulse wave amplitude and control the pressurizing element to continuously pressurize. When the processing device confirms that the pulse wave amplitude starts to decrease, the processing device controls the pressurizing element to depressurize until the pulse wave amplitude returns to a maximum amplitude pulse wave and records the pulse wave waveform of the radial artery under a constant pressure.

### CROSS-REFERENCE TO RELATED APPLICATION

This application claims the priority benefit of Taiwan application serial no. 112117709, filed on May 12, 2023. The entirety of the above-mentioned patent application is hereby incorporated by reference herein and made a part of this specification.

### BACKGROUND

### Technical Field

The disclosure relates to a measurement system, and particularly relates to an arterial pulse wave measurement system and a pulse wave measurement method.

### Description of Related Art

The traditional Chinese medicine pulse diagnosis is to use fingers to press three positions (i.e., three parts) of “cun”, “guan”, and “chi” on a wrist to obtain pulse manifestations, and observe physiological or pathological states of different organs and meridians based on the pulse manifestations of the three parts. Moreover, traditional Chinese pulse diagnosis instrument uses pressure sensors to perform measurement, and today's commonly used measurement method is to continuously pressurize the sensor on a radial artery, so that an amplitude of a blood pressure pulse wave gradually increases until a critical point, and then the amplitude of the blood pressure pulse wave gradually decreases to none. Due to a long pressurization time from continuous pressurization until the blood pressure pulse wave becomes small to none, an overall diagnosis time is affected, and when the sensor on the radial artery is pressurized to a fixed pressure value (for example, 200 mmHg (millimeter mercury column)) and then release the pressure, since a preset pressure value is usually high, it is easy to cause discomfort to the person being measured.

### SUMMARY

The disclosure is directed to an arterial pulse wave measurement system and a pulse wave measurement method, which are adapted to reduce a blood pressure pulse wave measurement time and reduce discomfort of a measured person.

The disclosure provides an arterial pulse wave measurement system including a first sensor, a pressurizing element, and a processing device. The first sensor is arranged on a radial artery to detect a pulse wave amplitude of the radial artery. The pressurizing element contacts the first sensor to pressurize the first sensor. The processing device is coupled to the first sensor and the pressurizing element to control the first sensor to continuously detect the pulse wave amplitude and control the pressurizing element to continuously pressurize. When the processing device confirms that the pulse wave amplitude starts to decrease, the processing device controls the pressurizing element to depressurize until the pulse wave amplitude returns to a maximum amplitude pulse wave and records a pulse wave waveform of the radial artery under a constant pressure.

The disclosure provides a pulse wave measurement method including following steps. A pulse wave amplitude of a radial artery is detected through a first sensor. The first sensor is pressurized by a pressurizing element. The pulse wave amplitude is continuously confirmed through a processing device and the pressurizing element is controlled to continuously pressurize. When the processing device confirms that the pulse wave amplitude starts to decrease, the processing device controls the pressurizing element to depressurize until the pulse wave amplitude returns to a maximum amplitude pulse wave and records a pulse wave waveform of the radial artery under a constant pressure.

Based on the above description, in the arterial pulse wave measurement system and the pulse wave measurement method of the embodiments of the disclosure, since the arterial pulse wave measurement system determines pressurization or depressurization based on a change of the pulse wave amplitude, it automatically adapt to the pulse wave characteristics of different people, and the maximum pressure may be applied to the corresponding maximum pressure point according to individual differences, i.e., the sensor will not be pressurized to a preset value and then depressurized, so that an overall measurement time will be shorter, and it may cause less discomfort to the person being diagnosed.

To make the aforementioned more comprehensible, several embodiments accompanied with drawings are described in detail as follows.

### BRIEF DESCRIPTION OF THE DRAWINGS

FIG. 1 is a system schematic diagram of an arterial pulse wave measurement system according to an embodiment of the disclosure.

FIG. 2 is a schematic waveform diagram of arterial pulse wave measurement according to an embodiment of the disclosure.

FIG. 3 is a flowchart of an arterial pulse wave measurement method according to an embodiment of the disclosure.

FIG. 4 is a schematic cross-sectional view of a pressurizing element according to an embodiment of the disclosure.

FIG. 5 is a system schematic diagram of an arterial pulse wave measurement system according to another embodiment of the disclosure.

FIG. 6 is a schematic waveform diagram of arterial pulse wave measurement according to another embodiment of the disclosure.

FIG. 7 is a flowchart of an arterial pulse wave measurement method according to another embodiment of the disclosure.

### DESCRIPTION OF THE EMBODIMENTS

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It should be noted that although the terms “first”, “second”, “third”, etc. may be used for describing various elements, components, regions, layers and/or portions, the elements, components, regions, layers and/or portions are not limited by these terms. These terms are only used for separating one element, component, region, layer or portion from another element, component, region, layer or portion. Therefore, the following discussed “first element”, “component”, “region”, “layer” or “portion” may be referred to as the second element, component, region, layer or portion without departing from the scope of the disclosure.

The terminology used herein is for the purpose of describing particular embodiments and is not intended to be limiting of the disclosure. As used herein, the singular forms “a,” “an” and “the” are intended to include the plural forms as well, unless the context clearly indicates otherwise. “or” represents “and/or”. The term “and/or” used herein includes any or a combination of one or more of the associated listed items. It will be further understood that the terms “comprises,” “comprising,” “includes” and/or “including,” when used herein, specify the presence of stated features, regions, integers, steps, operations, elements, and/or components, but do not preclude the presence or addition of one or more other features, regions, integers, steps, operations, elements, components, and/or groups thereof.

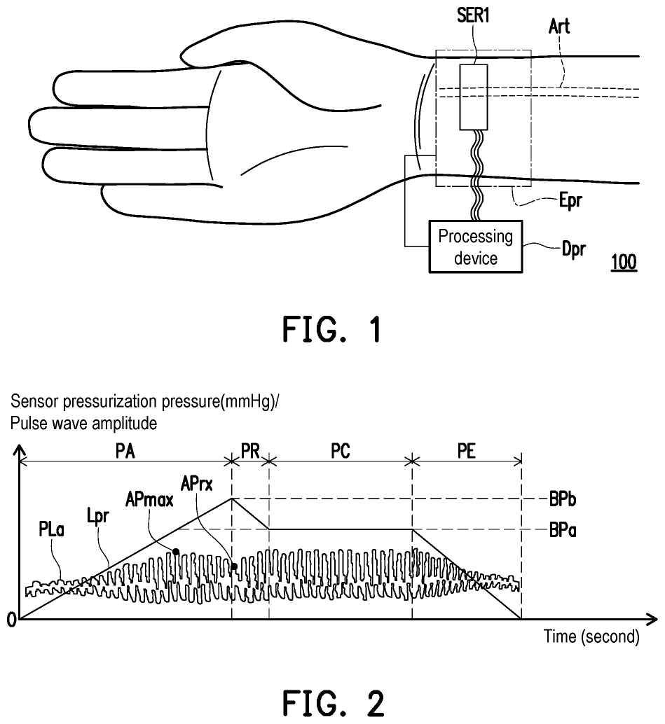

FIG. 1 is a system schematic diagram of an arterial pulse wave measurement system according to an embodiment of the disclosure. FIG. 2 is a schematic waveform diagram of arterial pulse wave measurement according to an embodiment of the disclosure. Referring to FIG. 1, in the embodiment, an arterial pulse wave measurement system 100 includes a first sensor SER1, a pressurizing element Epr, and a processing device Dpr.

The first sensor SER1 is disposed on a radial artery Art to detect a pulse wave amplitude of the radial artery Art. The pressurizing element Epr contacts the first sensor SER1 to pressurize the first sensor SER1. The processing device Dpr is coupled to the first sensor SER1 and the pressurizing element Epr, so as to control the first sensor SER1 to continuously detect the pulse wave amplitude and control the pressurizing element Epr to continuously pressurize.

Referring to FIG. 1 and FIG. 2, when the user is to be measured, the processing device Dpr controls the pressurizing element Epr to pressurize the first sensor SER1 (shown as a pressurization period PA), so that a pressure of the first sensor SER1 keeps increasing (shown as a waveform Lpr), and controls the first sensor SER1 to start detecting a pulse wave amplitude of the radial artery Art (shown as a waveform PLa). Then, when the processing device Dpr confirms that the pulse wave amplitude starts to decrease to a predetermined threshold (shown as a preset pulse wave amplitude APrx under a second pressure value BPb), the processing device Dpr controls the pressurizing element Epr to depressurize until the pulse wave amplitude returns to a maximum amplitude pulse wave APmax (i.e., during a pressure release period PR, the pressure is released to a first pressure value BPa) and recording of a pulse wave waveform of the radial artery Art is performed under a constant pressure (i.e., the waveform PLa during a constant pressure period PC). After completing the recording of the pulse wave waveform of the radial artery Art, the processing device Dpr controls the pressurizing element Ep to completely release the pressure (shown as a complete pressure release period PE).

According to the above description, since the arterial pulse wave measurement system 100 determines pressurization or depressurization according to a change of the pulse wave amplitude, i.e., the first sensor SER1 will not be pressurized to a preset value and then release the pressure, an overall measurement time may be shorter, and less discomfort is caused to the person being measured.

In the embodiment of the disclosure, the processing device Dpr may control to release the pressure until the pulse wave amplitude returns to the maximum amplitude pulse wave APmax when the pressurizing element Epr pressurizes until the pulse wave amplitude decreases to between 70%-80% of the maximum amplitude pulse wave APmax. In the embodiment of the disclosure, the record of the pulse wave waveform PLa of the radial artery Art (i.e., a time length of the constant pressure period PC) is maintained for at least 6 seconds. The above is an example for illustration, and the embodiment of the disclosure is not limited thereto.

In the embodiment of the disclosure, the first sensor SER1 may be a single sensing element (such as a piezoelectric element), or an array formed by a plurality of sensing elements. Moreover, the pressurizing device may be composed of a pump and an air bag, or may be composed of a stepping motor and a buffer pressure head (such as a relatively large rather than wrist type pulse diagnosis instrument). The above is an example for illustration, and the embodiment of the disclosure is not limited thereto.

In the embodiment of the disclosure, the processing device Dpr may be an electronic device having a processor and/or a circuit capable of executing software, but the embodiment of the disclosure is not limited thereto.

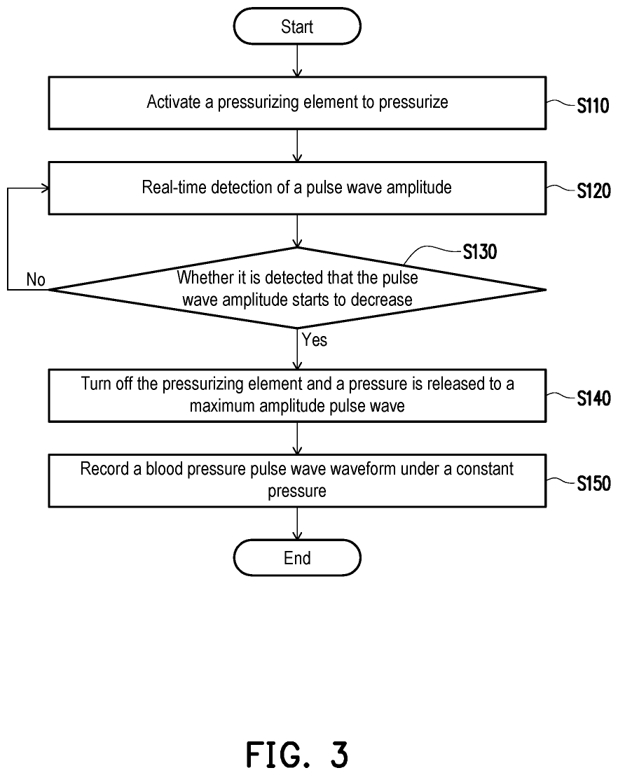

FIG. 3 is a flowchart of an arterial pulse wave measurement method according to an embodiment of the disclosure. Referring to FIG. 3, in the embodiment, the arterial pulse wave measurement method includes following steps. In step S110, a pressurizing element is activated to pressurize a first sensor. In step S120, real-time detection of a pulse wave amplitude is performed through the first sensor, i.e., a pulse wave amplitude of the radial artery is detected through the first sensor, where the processing controls to continuously detect the pulse wave amplitude and controls the pressurizing element to continuously pressurize. In step S130, the processing device determines whether it is detected that the pulse wave amplitude starts to decrease.

When it is not detected that the pulse wave amplitude starts to decrease, i.e., the determination result of step S130 is “No”, the process returns to step S120 to continuously detect the pulse wave amplitude. When it is detected that the pulse wave amplitude starts to decrease, i.e., the determination result of step S130 is “Ycs”, step S140 is executed. In step S140, the pressurizing element is turned off and the pressure is released to the maximum amplitude pulse wave. In step S150, a blood pressure pulse wave waveform is recorded under the constant pressure through the processing device, i.e., the processing device controls the pressurizing element to pressurize until the pulse wave amplitude returns to the maximum amplitude pulse wave, and recording of the pulse wave waveform of the radial artery pulse is performed under the constant pressure. Where, the order of steps S110, S120, S130, S140, and S150 is for illustration, and the embodiment of the disclosure is not limited thereto. Moreover, for details of steps S110, S120, S130, S140, and S150, reference may be made to the embodiment shown in FIG. 1 and FIG. 2, which will not be repeated here.

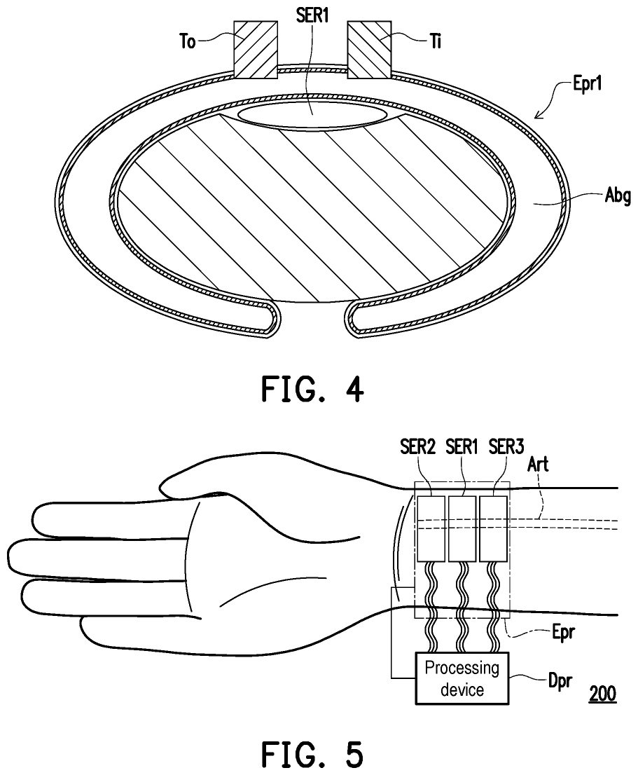

FIG. 4 is a schematic cross-sectional view of a pressurizing element according to an embodiment of the disclosure. Referring to FIG. 1 and FIG. 4, in the embodiment, the pressurizing element Epr may be, for example, the pressurizing element Epr1, where the pressurizing element Epr1 includes, for example, an airbag Abg, an inflation pump Ti, and a deflation pump To. The airbag Abg may wrap the first sensor SER1 and a wrist to pressurize the first sensor SER1, the inflation pump Ti is used to inflate the airbag Abg, and the deflation pump To is used to deflate the airbag Abg.

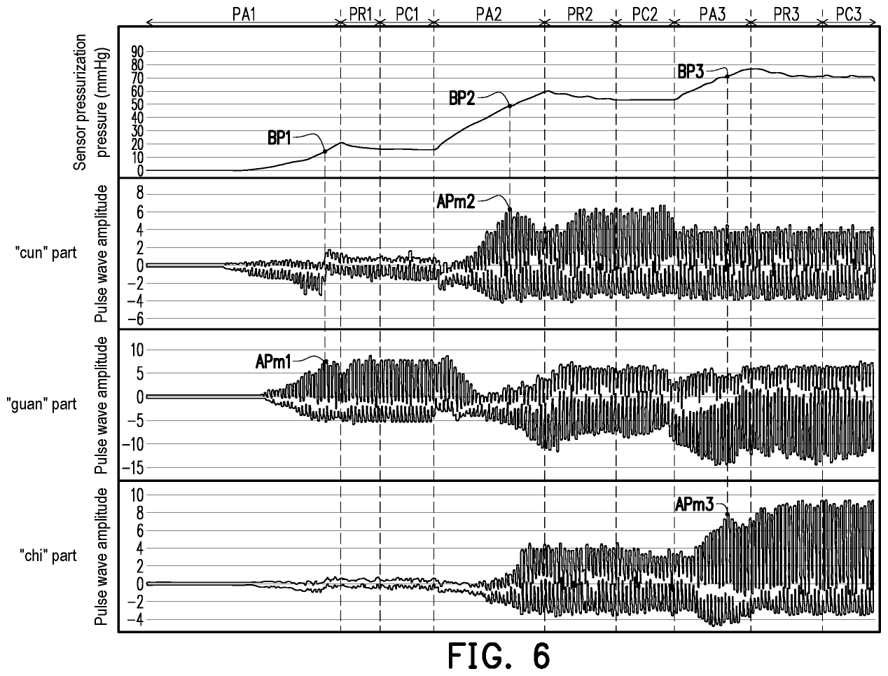

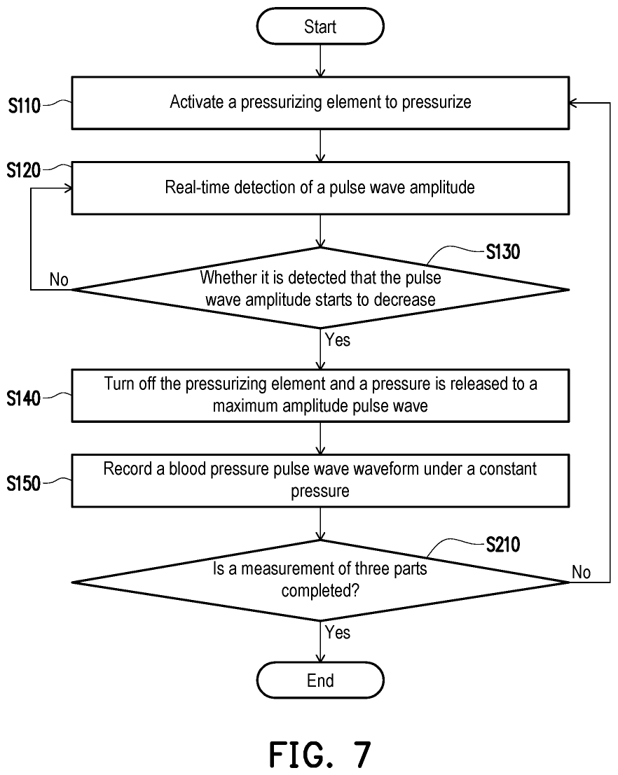

FIG. 5 is a system schematic diagram of an arterial pulse wave measurement system according to another embodiment of the disclosure. FIG. 6 is a schematic waveform diagram of arterial pulse wave measurement according to another embodiment of the disclosure. Referring to FIG. 1 and FIG. 5, in the embodiment, an arterial pulse wave measurement system 200 is substantially the same as the arterial pulse wave measurement system 100, and a difference there between is that the arterial pulse wave measurement system 200 further includes a plurality of sensors (such as a first sensor SER1, a second sensor SER2, and a third sensor SER3), where the same or similar components are denoted by the same or similar symbols.

In the embodiment, the second sensor SER2, the first sensor SER1, and the third sensor SER3 may be individually placed at positions of the three parts of the wrist “cun”, “guan”, and “chi” to measure waveforms of the pulse wave amplitudes of the three parts “cun”, “guan”, and “chi”. Moreover, the processing device Dpr may simultaneously confirm the multiple pulse wave amplitudes detected by the sensors SER1-SER3.

Referring to FIG. 5 and FIG. 6, when the user is to be measured, the processing device Dpr controls the pressurizing element Epr to simultaneously pressurize the first sensor SER1, the second sensor SER2 and the third sensor SER3 (shown as the pressurization period PA), so that pressures of the first sensor SER1, the second sensor SER2 and the third sensor SER3 keep increasing. Moreover, the processing device Dpr simultaneously confirms whether the pulse wave amplitudes of the three parts “cun”, “guan”, and “chi” start to decrease.

In the embodiment, it is assumed that the processing device Dpr first confirms that the pulse wave amplitude at the “guan” part starts to decrease. At this time, when the processing device Dpr confirms that the pulse wave amplitude of the “guan” part starts to decrease, the processing device Dpr controls the pressurizing element Epr to depressurize the first sensor SER1, the second sensor SER2, and the third sensor SER3 at the same time until the pulse wave amplitude returns to a maximum amplitude pulse wave APm1 (i.e., during a pressure release period PR1, the pressure is released to a pressure value BP1) and recording of a pulse wave waveform of the radial artery Art of the “guan” part is performed under the constant pressure (shown as a constant pressure period PC1). After completing the recording of the pulse wave waveform of the radial artery Art of the “guan” part, the processing device Dpr controls the pressure element Epr to again simultaneously pressurize the first sensor SER1, the second sensor SER2, and the third sensor SER3 (shown as a pressurization period PA2), but the processing device Dpr does not confirm the change of the pulse wave amplitude of the “guan” part to avoid repeated recording of the pulse wave waveform.

In the embodiment, it is assumed that the processing device Dpr then confirms that the pulse wave amplitude at the “cun” part starts to decrease. At this time, when the processing device Dpr confirms that the pulse wave amplitude of the “cun” part starts to decrease, the processing device Dpr controls the pressurizing element Epr to again depressurize the first sensor SER1, the second sensor SER2, and the third sensor SER3 at the same time until the pulse wave amplitude returns to a maximum amplitude pulse wave APm2 (i.e., during a pressure release period PR2, the pressure is released to a pressure value BP2) and recording of a pulse wave waveform of the radial artery Art of the “cun” part is performed under the constant pressure (shown as a constant pressure period PC2). After completing the recording of the pulse wave waveform of the radial artery Art of the “cun” part, the processing device Dpr controls the pressure element Epr to again simultaneously pressurize the first sensor SER1, the second sensor SER2, and the third sensor SER3 (shown as a pressurization period PA3), but the processing device Dpr does not confirm the changes of the pulse wave amplitudes of the “guan” part and the “cun” part to avoid repeated recording of the pulse wave waveforms.

In the embodiment, it is assumed that the processing device Dpr finally confirms that the pulse wave amplitude at the “chi” part starts to decrease. At this time, when the processing device Dpr confirms that the pulse wave amplitude of the “chi” part starts to decrease, the processing device Dpr controls the pressurizing element Epr to again depressurize the first sensor SER1, the second sensor SER2, and the third sensor SER3 at the same time until the pulse wave amplitude returns to a maximum amplitude pulse wave APm3 (i.e., during a pressure release period PR3, the pressure is released to a pressure value BP3) and recording of a pulse wave waveform of the radial artery Art of the “chi” part is performed under the constant pressure (shown as a constant pressure period PC3). After completing the recording of the pulse wave waveform of the radial artery Art of the “chi” part, the processing device Dpr controls the pressurizing element Epr to completely depressurize the first sensor SER1, the second sensor SER2, and the third sensor SER3 at the same time.

According to the above description, the first sensor SER1, the second sensor SER2, and the third sensor SER3 placed on the three parts of “cun”, “guan”, and “chi” of the wrist all perform depressurization and a constant pressure process at the same time, and only record the maximum amplitude pulse wave waveform. Then, the first sensor SER1, the second sensor SER2, and the third sensor SER3 are pressurized again and the above steps are repeated until all the pulse wave waveforms of the sensors are recorded. In other words, when the pulse wave amplitude of each of these sensors SER1-SER3 starts to decrease, the processing device Dpr controls the pressurizing element Epr to depressurize these sensors SER1-SER3 at the same time, so that these pulse wave amplitudes respectively return to the corresponding maximum amplitude pulse waves (such as APm1-APm3), and the plurality of pulse wave waveforms of the radial artery Art sensed by the sensors SER1-SER3 at corresponding maximum amplitude pulse waves (such as APm1-APm3) are recorded under constant pressure.

FIG. 7 is a flowchart of an arterial pulse wave measurement method according to another embodiment of the disclosure. Referring to FIG. 3 and FIG. 7, a process flow of the arterial pulse wave measurement method in FIG. 7 is roughly the same as the process flow of the arterial pulse wave measurement method in FIG. 3, and a difference there between is that the arterial pulse wave measurement method in FIG. 7 further includes step S210. In step S210, the processing device Dpr determines whether measurement (i.e., recording of the pulse wave waveforms) of the three parts of “cun”, “guan”, and “chi” of the wrist is completed.

When the measurement of the three parts of “cun”, “guan”, and “chi” is not completed, i.e., the determination result of step S210 is “no”, the process returns to step S110 to pressurize the first sensor SER1, the second sensor SER2, and the third sensor SER3 at the same time; when the measurement of “cun”, “guan”, and “chi” has been completed, i.e., the determination result of step S210 is “yes”, then the pulse wave measurement method ends. Where, the order of steps S110, S120, S130, S140, S150, and S210 is for illustration, and the embodiment of the disclosure is not limited thereto. Moreover, for details of steps S110, S120, S130, S140, S150, and S210, reference may be made to the embodiments shown in FIG. 5 and FIG. 6, which will not be repeated here.

In summary, in the arterial pulse wave measurement system and the pulse wave measurement method of the embodiments of the disclosure, since the arterial pulse wave measurement system determines pressurization or depressurization based on a change of the pulse wave amplitude, it automatically adapt to the pulse wave characteristics of different people, and the maximum pressure may be applied to the corresponding maximum pressure point according to individual differences, i.e., the sensor will not be pressurized to a preset value and then depressurized, so that an overall measurement time will be shorter, and it may cause less discomfort to the person being diagnosed.

It will be apparent to those skilled in the art that various modifications and variations can be made to the disclosed embodiments without departing from the scope or spirit of the disclosure. In view of the foregoing, it is intended that the disclosure covers modifications and variations provided they fall within the scope of the following claims and their equivalents.

What is claimed is:

1. An arterial pulse wave measurement system, comprising:
a first sensor, arranged on a radial artery to detect a pulse wave amplitude of the radial artery;
a pressurizing element, contacting the first sensor to pressurize the first sensor;
a processing device, coupled to the first sensor and the pressurizing element to control the first sensor to continuously detect the pulse wave amplitude and control the pressurizing element to continuously pressurize,
wherein the processing device controls the pressurizing element to depressurize until the pulse wave amplitude returns to a maximum amplitude pulse wave and records a pulse wave waveform of the radial artery under a constant pressure when the processing device confirms that the pulse wave amplitude starts to decrease.

2. The arterial pulse wave measurement system according to claim 1, wherein the processing device releases a pressure until the pulse wave amplitude returns to the maximum amplitude pulse wave when the pressurizing element pressurizes until the pulse wave amplitude decreases to between 70%-80% of the maximum amplitude pulse wave.

3. The arterial pulse wave measurement system according to claim 1, wherein a record of the pulse wave waveform of the radial artery is maintained for at least 6 seconds.

4. The arterial pulse wave measurement system according to claim 1, further comprising a plurality of sensors comprising the first sensor.

5. The arterial pulse wave measurement system according to claim 4, wherein the sensors comprise three sensors.

6. The arterial pulse wave measurement system according to claim 5, wherein the three sensors measure three parts of “cun”, “guan”, and “chi”.

7. The arterial pulse wave measurement system according to claim 4, wherein the processing device simultaneously confirms a plurality of pulse wave amplitudes detected by the sensors, and
the processing device controls the pressurizing element to simultaneously depressurize the sensors such that the pulse wave amplitudes respectively return to the corresponding maximum amplitude pulse wave and records a plurality of pulse wave waveforms of the radial artery sensed by the sensors at the corresponding maximum amplitude pulse wave under the constant pressure when the pulse wave amplitude of each of the sensors starts to decrease.

8. An arterial pulse wave measurement method, comprising:
detecting a pulse wave amplitude of a radial artery by a first sensor;
pressurizing the first sensor by a pressurizing element;
continuously confirming the pulse wave amplitude and controlling the pressurizing element to continuously pressurize by a processing device; and
controlling the pressurizing element to depressurize until the pulse wave amplitude returns to a maximum amplitude pulse wave and recording a pulse wave waveform of the radial artery under a constant pressure by the processing device when the processing device confirms that the pulse wave amplitude starts to decrease.

9. The arterial pulse wave measurement method according to claim 8, wherein the processing device releases a pressure until the pulse wave amplitude returns to the maximum amplitude pulse wave when the pressurizing element pressurizes until the pulse wave amplitude decreases to between 70%-80% of the maximum amplitude pulse wave.

10. The arterial pulse wave measurement method according to claim 8, further comprising:
detecting a plurality of pulse wave amplitudes of the radial artery at a plurality of parts by a plurality of sensors comprising the first sensor,
wherein the processing device controls the pressurizing element to simultaneously depressurize the sensors such that the pulse wave amplitudes respectively return to the corresponding maximum amplitude pulse wave and records a plurality of pulse wave waveforms of the radial artery sensed by the sensors at the corresponding maximum amplitude pulse wave under the constant pressure when the pulse wave amplitude of each of the sensors starts to decrease.
